(19) United States
(12) Patent Application Publication
(10) Pub. No.: US 2009/0024197 A1
(43) Pub. Date: Jan. 22, 2009

(54) ELUTION CONTROL VIA GEOMETRIC FEATURES OF AN IMPLANTABLE SUBSTANCE MATRIX

(75) Inventor: Robert Oskar Jensen, New Brighton, MN (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/779,652

(22) Filed: Jul. 18, 2007

(51) Int. Cl. A61N 1/05

(52) U.S. Cl. 607/120

(57) ABSTRACT

A cardiac lead that is adapted and configured to elute a therapeutic agent to treat the surrounding tissue at a target location within a patient's heart is described. The cardiac lead includes a drug eluting member having a predetermined number of macroscopic surface features formed on its exterior surface. The macroscopic surface features allow the elution rate to be controlled and/or increased without the need for modifying the amount of therapeutic agent in the drug eluting member.

### TECHNICAL FIELD

The present invention is related to medical electric leads, and more particularly cardiac leads that are adapted and configured to elute a therapeutic agent to treat the surrounding tissue at a target location within a patient's heart.

### BACKGROUND

Implantable cardiac stimulation leads are well known in the art. In general, these devices have an elongated flexible body with an electrode at one end for contacting cardiac tissue and a connector at the other end for mating with an automated stimulation device, namely a pacemaker or defibrillator. Depending upon the type of therapy to be delivered, the cardiac lead can be placed on either the right or the left side of the heart. The electrode of a cardiac lead then may be secured at a target location within the heart by active or passive fixation.

When a cardiac lead has been implanted in the heart it has been determined that the cardiac tissue at the site of implantation will react favorably to the lead in the presence of a therapeutic drug, such as, for example, a steroid. Consequently, cardiac leads have been designed with means, such as a collar, for delivering a therapeutic drug to the cardiac tissue at the implantation site. Overcoming slow drug elution rates resulting in low amounts of drug delivered is one of the challenges associated with current drug eluting cardiac lead products.

### SUMMARY

According to one embodiment of the present invention, a cardiac lead includes a conductive lead body having a proximal end and a distal end and at least one electrode located on the lead body, and a drug eluting member adjacent to at least one electrode. The cardiac lead also includes drug eluting member having an exterior surface adjacent to the at least one electrode. The drug eluting member comprises a mixture comprising of at least one polymer and a therapeutic agent. The exterior surface of the drug eluting member includes a plurality of macroscopic surface features arranged in a non-random, ordered pattern.

According to another embodiment of the present invention, a cardiac lead includes a conductive lead body including a proximal end and a distal end, at least one electrode located on the lead body, and a drug eluting member having an exterior surface adjacent the at least one electrode. The drug eluting member is formed from a mixture comprising a non-biodegradable polymer and a therapeutic agent. The cardiac lead also includes a means for increasing the surface area of the drug eluting member having a major size dimension ranging from about 0.001 inches to about 0.10 inches. The means for increasing the surface area of the drug eluting member includes a non-random, ordered pattern of holes, blind holes, nubs, bumps, generally cylindrical protrusions, dimples, slots, channels, ridges, generally rectangular ridges and combinations thereof formed on the exterior surface of the drug eluting member.

According to yet another embodiment, the present invention is a method of forming a drug eluting member configured to control an elution rate of a therapeutic agent. The method includes providing a mold tool having a plurality of mold features configured to form a plurality of corresponding macroscopic surface features on an exterior surface of the drug eluting member; providing a mixture including a non-biodegradable polymer and the therapeutic agent; and using the mold tool to mold the drug eluting member from the polymer mixture such that the drug eluting member comprises a plurality of macroscopic surface features formed on the exterior surface of the drug eluting member. The macroscopic surface features are arranged on the exterior surface of the drug eluting member in a non-random, ordered pattern.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of a cardiac lead according to an embodiment of the present invention.

FIG. 2 is a close-up perspective view of a distal end of the cardiac lead shown in FIG. 1 according to an embodiment of the present invention.

FIGS. 3A-3D are close-up schematic views of a drug eluting member according to various embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

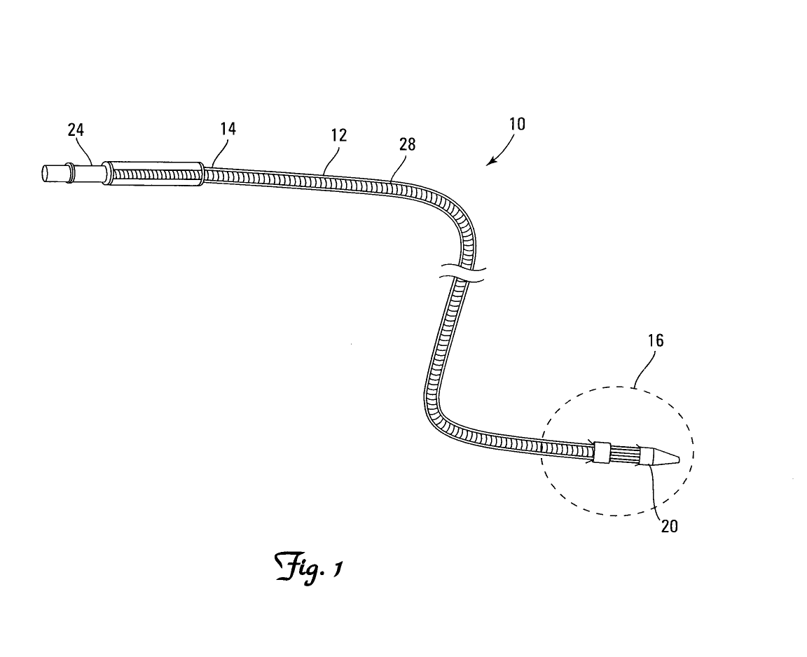

FIG. 1 shows a cardiac lead 10 according to an embodiment of the present invention. Cardiac lead 10 includes an elongated conductive lead body 12 having opposed proximal and distal ends 14 and 16. The lead body 12 is formed from a bio-compatible insulative material such as silicone rubber, polyurethane or the like. At least one electrode 20 with an annular contact surface is operatively associated with the distal end 16 of the lead body 12. Alternatively, the electrode 20 can be located anywhere along the lead body 12. The electrode 20 is coated with or formed from platinum, stainless steel, MP35N, a platinum-iridium alloy or another similar conductive material. A connector 24 is operatively associated with the proximal end 14 of the lead body 12. The connector 24 may be of any standard type, size or configuration. Connector 24 is electrically connected to the ring electrode 20 by way of a conductor coil 28 that extends through the interior lumen of lead body 12. Conductor coil 28 is generally helical in configuration and includes one or more conductive wires or filaments. For example, the conductor may be a multifilar conductor coil with as many as eight filaments. According to some embodiments, the conductor or conductors can be coupled to one or more electrodes such as in a bipolar, tripolar, or quadrupolar pacing lead. In yet a further embodiment of the present invention, the cardiac lead 10 includes a lumen for receiving a guiding element such as a guide wire or a stylet.

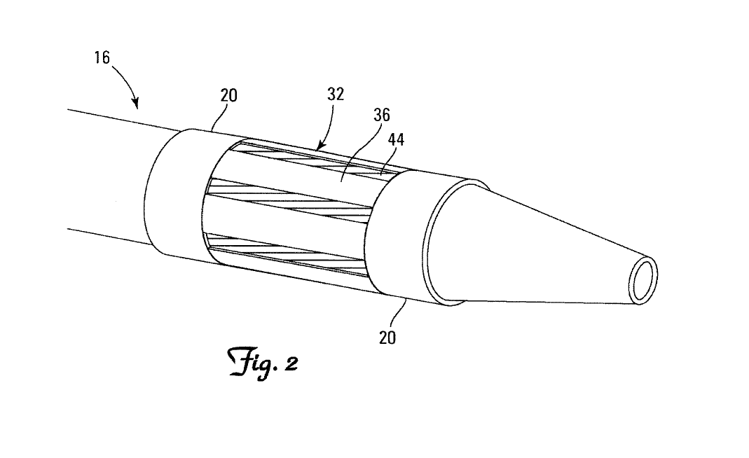

As shown in FIG. 2, a drug eluting member 32 is disposed at the distal end 16 of lead body 12 adjacent to or surrounding a portion of an electrode 20. The drug eluting member 32 is a sheath or a collar that is formed from a mixture that includes at least one non-biodegradable polymer and at least one therapeutic agent. According to one embodiment, the drug eluting member has an axial length ranging from about 0.03 Inches to about 0.10 Inches.

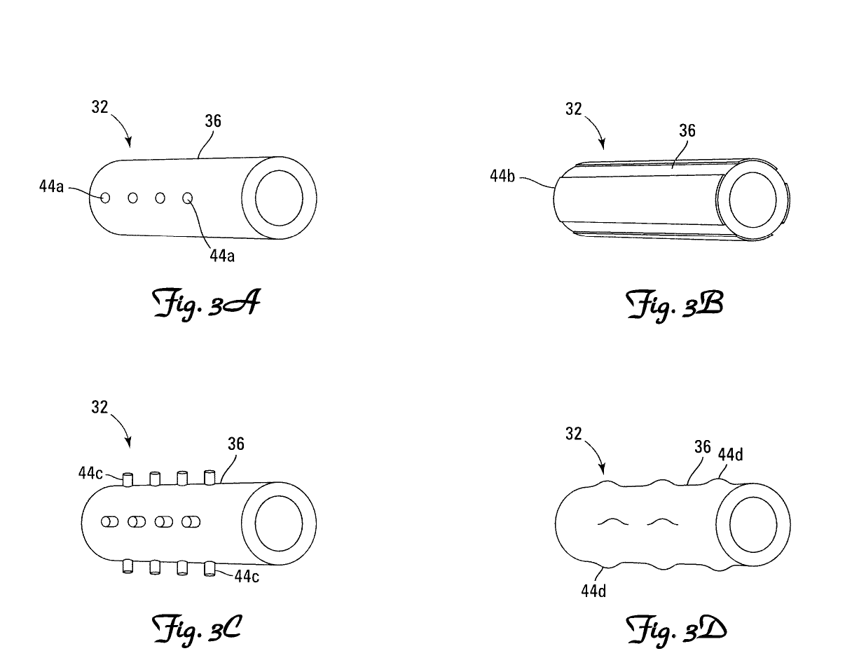

As further shown in FIG. 2, as well as in FIGS. 3A-3D, the drug eluting member 32 includes an exterior surface 36 having a plurality of macroscopic surface features 44 that are adapted to be exposed to and in contact with the surrounding environment, such as cardiac tissue or vascular tissue, when the lead is delivered to a target location within a patient's heart. The macroscopic surface features 44 included on the exterior surface 36 are formed during formation of the drug eluting member 32. The macroscopic surface features 44 form a non-random, ordered pattern on the exterior surface 36 of the drug eluting member 32.

One way to increase the elution rate of a therapeutic agent from a drug eluting member is to alter the mass fraction of the therapeutic agent in the drug eluting member matrix by increasing the amount of therapeutic agent provided in the matrix. An alternative solution to altering the mass fraction ratio of the drug eluting matrix is to increase the amount of surface area of the drug eluting member that comes into contact with the surrounding environment. According to one embodiment of the present invention, the macroscopic surface features 44 increase the amount of surface area that comes into contact with the surrounding environment.

The elution of a therapeutic agent from a drug eluting member, as described above, is dependent upon the amount of surface area that comes into contact with the surrounding environment (e.g. tissue, bodily fluid, etc.). Additionally, the elution of the therapeutic agent(s) may decline or cease once the therapeutic agent has been dissipated from the exposed matrix surface. Accordingly, by controlling the amount of exposed surface area that comes into contact with the surrounding environment, the elution rate and/or the total amount of therapeutic agent delivered (total dosage amount) can be manipulated and/or controlled. Thus, the number, type, and size of macroscopic surface features can be used to control and manipulate the rate of elution of the therapeutic agent into the surrounding environment.

Exemplary types of macroscopic surface features include, but are not limited to, the following: holes, blind holes (e.g. holes having a specified depth), nubs, bumps, generally cylindrical protrusions, dimples, slots, channels, ridges, generally rectangular ridges and combinations thereof. FIG. 3A shows an exemplary drug eluting member 32 having four holes 44a formed in the exterior surface 36. FIG. 3B shows another exemplary drug eluting member 32 having a number of channels 44b formed in the exterior surface 36. FIG. 3C shows yet another exemplary embodiment of a drug eluting member 32 having a plurality of generally cylindrical protrusions 44c formed on the exterior surface 36. Finally, FIG. 3D is yet another exemplary embodiment of the drug eluting member 32 having a plurality of bumps or nubs 44d formed on the exterior surface 36.

The macroscopic surface features are fewer in number and larger in dimension (e.g., length, width, height, depth, or diameter) than microscopic surface features (micropores, surface roughness, etc) or mesoporous surface features. Microscopic surface features typically have a major size dimension of less than 2 nm. Mesoporous surface features typically have a major size dimension ranging from about 2 nm to about 50 nm. Macroscopic surface features have a major size dimension greater than 50 nm. The term “major size dimension” refers to the primary, largest, or most prominent dimension of a given surface feature, such as the length, width, height, depth or diameter of the feature. For example, the major size dimension of a generally cylindrical protrusion may be its height or diameter dimension. The major size dimension of a ridge may be its length dimension. Finally, the major size dimension of a hole may be its depth or diameter dimension. In one embodiment, the major dimension of the macroscopic feature is the feature's largest dimension.

According to one embodiment of the present invention, the macroscopic surface features formed on the exterior surface of the drug eluting member 32 have a major size dimension of at least 0.001 inches. That is, the macroscopic surface features formed on the exterior surface of the drug eluting member can have an approximate length, height, depth, width, or diameter of at least 0.001 inches. According to another exemplary embodiment of the present invention, the macroscopic surface features formed on the exterior surface of the drug eluting member have a major size dimension ranging from about 0.001 inches to about 0.10 inches. For example, a macroscopic surface feature may have an approximate length, height, depth, or outer diameter ranging from about 0.001 inches to about 0.10 inches.

Typically, the number of macroscopic surface features formed on the exterior surface 36 of a given drug eluting member 32 ranges from about 2 to about 16. According to one exemplary embodiment, the number of macroscopic surface features ranges from about 4 to about 9.

The macroscopic surface features 44a-44d are formed on the surface 36 of the drug eluting member 32 during the formation of the drug eluting member 32 such that they form a non-random, ordered pattern on the exterior surface 36. For example, as shown in FIG. 3C, a plurality of generally cylindrical protrusions 44c may be formed on the surface 36. The generally cylindrical protrusions 44c are provided in a plurality of rows, each row spaced equidistant from one another around the circumference of the drug eluting member 32. Within each row, each cylindrical protrusion 44c is spaced at an equal distance from the next. According to another exemplary embodiment, four holes of a specified depth may be formed in the exterior surface 36 of the drug eluting member 32. The holes can be provided at ninety degree points about the circumference of the drug eluting member 32. In one embodiment, an ordered pattern of macroscopic features is provided across all or a substantial portion of the drug eluting member 32.

The incorporation of a non-biodegradable polymer into the drug eluting member helps to control the dissolution/elution of the therapeutic agent into the surrounding environment. Additionally, the rate of diffusion of the therapeutic agent through the non-biodegradable polymer may be affected by drug solubility, polymer hydrophilicity, extent of polymer cross-linking, expansion of the polymer upon water absorption, and the like.

Exemplary biocompatible, non-biodegradable polymers used to form the drug eluting member 32 include, but are not limited to, the following: poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(-vinylidene chloride), poly(acrylonitrile), cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloridediethyl fumarate copolymer, silicone, silicone rubbers, poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls), poly(acrylate), poly(methacrylate), poly(oxides), poly(esters), poly(amides), and poly(carbonates). According to one embodiment of the present invention, the non-biodegradable polymer is silicone rubber.

Many different types of therapeutic agents can be incorporated into the mixture used to form the drug eluting member 32. In many embodiments, the therapeutic agent is a steroid. In use, the therapeutic agent, or steroid elultes from the polymer matrix over time having a desirable effect on surrounding cardiac tissue. Exemplary steroids include, but are not limited to, the following: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

According to one exemplary embodiment of the present invention, the therapeutic agent is dexamethasone sodium phosphate or alternatively, dexamethasone acetate.

According to an embodiment of the present invention, the drug eluting member 32 is formed by mixing liquid silicone rubber (LSR) together with a steroid. The amount of steroid includes in the polymer matrix ranges from about 15% to about 70%. According to another exemplary embodiment of the present invention the amount of steroid included in the polymer matrix ranges from about 30% to about 40%. According to a further embodiment, the steroid is dexamethasone sodium acetate. The composition is then molded into a tubular form using a mold tool that includes a number of mold features that correspond to the macroscopic surface features to be formed on the exterior surface 36 of the drug eluting member 32. Different mold tools can be selected depending upon the type, size, number, and pattern of macroscopic surface features desired on the exterior surface 36 of the drug eluting member 32. The tube is then cut into collars or sheaths having a desired length. According to one alternative embodiment, the drug eluting collar can be extruded.

According to another alternative embodiment, a masking process can be used to etch or otherwise form the macroscopic surface features on the exterior surface 36 of the drug eluting member 32. Whatever the process, after formation, the collar or sheath is then bonded in place on the lead body using a silicone adhesive.

According to a further exemplary embodiment of the present invention, the drug eluting member 32 is molded from a mixture containing a two-part platinum cured silicone rubber, and one part dexamethasone acetate. According to this embodiment, the polymer matrix is approximately 33% liquid silicone rubber A, 33% liquid silicone rubber B, and 33% dexamethasone sodium acetate. The elution rate of dexamethasone from the polymer matrix can be manipulated by the number, type, size, and pattern of macroscopic surface features formed on the exterior surface 36 of the drug eluting member 32.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fail within the scope of the claims, together with all equivalents thereof.

I claim:

1. A cardiac lead, the lead comprising:
a conductive lead body including a proximal end and a distal end;
at least one electrode located on the lead body; and
a drug eluting member having an exterior surface adjacent the at least one electrode, wherein the drug eluting member comprises a mixture of at least one polymer and a therapeutic agent, and wherein the exterior surface of the drug eluting member includes a plurality of macroscopic surface features arranged in a non-random pattern.

2. The cardiac lead according to claim 1, wherein the macroscopic surface features comprise a plurality of bumps formed on the exterior surface of the drug eluting member.

3. The cardiac lead according to claim 1, wherein the macroscopic surface features comprise a plurality of holes formed on the exterior surface of the drug eluting member.

4. The cardiac lead according to claim 1, wherein the macroscopic surface features comprise a plurality of generally cylindrical protrusions formed in the exterior surface of the drug eluting member.

5. The cardiac lead according to claim 1, wherein the macroscopic surface features comprise a plurality of channels formed in the exterior surface of the drug eluting member.

6. The cardiac lead according to claim 1, wherein the macroscopic surface features have a major size dimension of at least 0.001 inches.

7. The cardiac lead according to claim 1, wherein the macroscopic surface features have a major size dimension ranging from about 0.001 inches to about 0.10 inches.

8. The cardiac lead according to claim 1, wherein a number of macroscopic surface features ranges from about 4 to about 9.

9. The cardiac lead according to claim 1, wherein the mixture comprises about 15% to about 70% steroid.

10. The cardiac lead according to claim 1, wherein the mixture comprises about 30% to about 40% steroid.

11. The cardiac lead according to claim 1, wherein the mixture comprises about 30% to about 40% dexamethasone acetate.

12. The cardiac lead according to claim 1, wherein the polymer is silicone rubber.

13. A cardiac lead comprising:
a conductive lead body including a proximal end and a distal end;
at least one electrode located on the lead body;
a drug eluting member having an exterior surface adjacent the at least one electrode, wherein the drug eluting member is formed from a mixture comprising a non-biodegradable polymer and a therapeutic agent; and
a means for increasing the surface area of the drug eluting member, the means having a major size dimension ranging from about 0.001 inches to about 0.10 inches.

14. The cardiac lead according to claim 13, wherein the means for increasing the surface area of the drug eluting member comprise a non-random, ordered pattern of holes, blind holes, nubs, bumps, generally cylindrical protrusions, dimples, slots, channels, ridges, generally rectangular ridges and combinations thereof formed on the exterior surface of the drug eluting member.

15. The cardiac lead according to claim 13, wherein the mixture comprises about 30% to about 40% dexamethasone acetate.

16. The cardiac lead according to claim 13, wherein the non-biodegradable polymer is silicone rubber.

17. The cardiac lead according to claim 13, wherein the macroscopic surface features have a major size dimension of at least 0.001 inches.

18. The cardiac lead according to claim 13, wherein the macroscopic surface features have a major size dimension ranging from about 0.001 inches to about 0.10 inches.

19. The cardiac lead according to claim 13, wherein a number of macroscopic surface features ranges from about 4 to about 9.

20. A method of forming a drug eluting member configured to control an elution rate of a therapeutic agent, the method comprising:
providing a mold tool having a plurality of mold features configured to form a plurality of corresponding macroscopic surface features on an exterior surface of the drug eluting member;
providing a mixture comprising a polymer and a therapeutic agent; and
using the mold tool to mold the drug eluting member from the polymer mixture such that the drug eluting member comprises a plurality of macroscopic surface features formed on the exterior surface of the drug eluting member, wherein the macroscopic surface features are arranged in a non-random, ordered pattern.
